(19) United States
(12) Patent Application Publication
(10) Pub. No.: US 2018/0002374 A1
(43) Pub. Date: Jan. 4, 2018

(54) Non-Denatured Proteins Derived From a Biomass Source

(71) Applicant: Green Recovery Technologies, LLC, New Castle, DE (US)

(72) Inventor: Kenneth L. Laubsch, Mullica Hill, NJ (US)

(21) Appl. No.: 15/703,932

(22) Filed: Sep. 13, 2017

Related U.S. Application Data
(63) Continuation-in-part of application No. 15/457,450, filed on Mar. 13, 2017.
Application No. 15/457,450 is a continuation of application No. 13/804,466, filed on Mar. 14, 2013, now Pat. No. 9,651,304.

(51) Int. Cl. C07K 1/36; C11B 13/00; C11B 3/00; C11B 1/10; B01D 3/00; C02F 1/00; B01D 33/06; B01D 21/02; F26B 5/00; B01D 11/02; C11B 1/02; C02F 101/32

(52) U.S. Cl. CPC C07K 1/36; B01D 11/02; C11B 13/00; C11B 3/008; C11B 1/10; C11B 1/02; B01D 11/0288; B01D 3/00; C02F 1/00; B01D 33/06; B01D 21/10; B01D 11/0292; F26B 5/005; C02F 2101/32; C02F 2001/007

(57) ABSTRACT

A biomass-derived protein compound has a high concentration of protein and can be made to have a very low concentration of fat and water; even when the biomass feedstock has a high fat concentration. The biomass-derived protein compound may be a whole protein that is non-denatured and enzymatically digestible. This unique protein compound can be produced from molecules from more than one source organism, including various animals and/or plant feedstocks. The unique protein compound is derived from a unique biomass method and apparatus for the treatment of a biomass stream to extract and separate an essentially solvent-free product from the biomass stream. In this unique method the solids content of the biomass stream is increased by bringing the biomass stream into contact with a moderately pressurized liquefied gas solvent, to create a high solids content biomass stream and introducing the high solids content biomass stream to an extraction apparatus.

### CROSS-REFERENCE TO RELATED APPLICATION

The present application is a continuation in part of U.S. Utility application Ser. No. 15/457,450, filed on Mar. 13, 2017, entitled Continuous System and Process For a Low-Water Biomass Stream with Liquefied-Gas Solvent to Separate and Recover Organic Products, which is currently pending, which is a continuation of Ser. No. 13/804,446, filed Mar. 14, 2013, and now issued as U.S. Pat. No. 9,651,304; the entirety of both applications are hereby incorporated by reference herein.

### BACKGROUND OF THE INVENTION

### Field of the Invention

The invention relates to a method for dewatering and separating animal process wastewater, vegetable and fruit waste and other industrial and municipal waste and post-waste streams using ambient temperature mechanical, vacuum and chemical dewatering in the presence of a liquefied gas solvent in a filter system to yield one or more proteins, lipids and/or other useful biomass extracts.

### Description of the Related Technology

Various types of dewatering and separation processes are employed to recover materials dissolved or suspended in waste streams. Typical biomass streams consist of three types of water: bulk, interstitial and cellular. Many dewatering and separation methods, solvents, gases and apparatus exist to separate different types and volumes of biomass into useful by-products with a wide range of values, from commodities to value added ingredients. In the animal processing waste product industry, dissolved air flotation and other coagulant and flocculation processes with or without some combination with dewatering processes are used most often to separate saleable by-products, such as lipids, from the animal processing wastewater streams. However, much of the value in lipids and proteins remain in the post-coagulated and flocculated material commonly known as “DAF” (“DAF” is an acronym for “dissolved air flotation” and sometimes also is used in reference to the resulting material after dissolved air flotation processing). While poultry, beef, pork, dairy and fish waste streams respond differently to coagulation and flocculation and dewatering processes, significant quantities of valuable by-products still remain in the waste stream. By contrast, fruit, vegetable and other botanical matter may employ different processes for recovering saleable by-products which apply extensive heat and pressure to produce certain extracts, although the heat and pressure can damage the resultant products in ways that limit usefulness and therefore their value.

Others have described methods and systems to process “DAF” and other waste materials. For example, U.S. Pat. No. 7,186,796 provides a method of isolating a bio-molecule including peptides, proteins, polynucleotides and polysaccharides from a water-borne mixture by contacting the water-borne mixture with dimethyl ether to precipitate solid particles of the bio-molecule. The water-borne mixtures include aqueous solutions, suspensions, emulsions, micro-emulsions and liposomes suspended in aqueous media. Similarly, U.S. Pat. No. 7,897,050 provides a method and system for the extraction of an organic chemical constituent, including hydrocarbons, crude petroleum products, refined petroleum products, synthetic compounds from a solid matter, including from animal renderings, using an inclined auger in a pressurized chamber. Thus, prior art systems have focused on higher value input streams, and generally worked with smaller volumes of waste materials where the methods, apparatus and chemistry can yield a higher value output. Prior art has been limited by the cost to scale the process in the form of cost prohibitive capital equipment needed to process large volumes of waste streams and/or the operating cost in the form of energy and pressures required to separate the waste streams into valuable commodity products.

In principle, continuously and discontinuously operating pressing apparatuses, e.g. multi-platen presses, belt presses, strainer presses, plate filter presses, travelling screen presses and screw presses, are suitable for separating off the lipids from the biomass. Centrifuges are likewise suitable for separating off lipids from the biomass. Known types of centrifuges include, for example, turnout centrifuges, peeler centrifuges, pusher centrifuges, mesh screw centrifuges, vibrating centrifuges and sliding centrifuges and decanting centrifuges. See, e.g., WO 2010/001492 A 1, relating generally to recovering tallow and more particularly, to removing fats, oil and grease and recovering tallow from food or animal processing wastewater by adding a flocculant and separating the tallow from the solids employing a centrifuge.

A further method, which has attained importance for separating lipids and proteins from a biomass, is filtration. A distinction is made between discontinuous and continuous filtering systems. Discontinuously operating filters include, for example, fixed-bed filters, suction filters, candle filters, leaf filters and plate filters. The separation of lipids and protein from the biomass by means of discontinuously operating filters is generally less preferred. A disadvantage here is the loading and unloading of the filter, which requires a considerable time, as well as filter clogging related to lipid viscosity exacerbated by lower processing temperatures where liquefied gases are employed. It is a further disadvantage that DAF, in aqueous solution, yields flux rates that make traditional commercial filters unusable due to particle size distribution promoting filter blinding. Thus, discontinuously operating filters are not suitable for large biomass throughputs. Large biomass throughputs can be categorized as several tons of input waste per hour.

Continuously operating filters, such as belt filters and rotary filters, also have been found to be useful as separation apparatuses, and rotary pressure filters as are known from WO 02/100512 A1 are particularly suitable.

For example, U.S. Pat. No. 5,162,129 to Anderson et al., provides a method of isolating proteins from waste raw animal parts. As described, grinding is utilized to break down the proteins which can result in smaller chain proteins. In addition, mechanical dewatering is described in this method which is an energy intensive process and unless done at low temperatures will denature the proteins. The method in Anderson et al. further describes the use of heat to stop the activity of enzymes which can denature the proteins.

It is well known that proteins exposed to high temperatures will become denatured. Protein denaturing is a change in the structure of the protein that can be caused by chemical effects or exposure to high temperatures. A denatured protein is a protein in which the amino acid composition and stereochemical structure (shape) have been altered by physical or chemical means. Denaturation is a process in which protein molecules or nucleic acids lose the quaternary structure, tertiary structure and secondary structure which is present in their native state, by application of some external stress or compound. When a protein molecule is denatured, secondary and tertiary structures are altered but the peptide bonds of the primary structure between the amino acids are left intact. Denaturing changes the protein structure and can change the flavor of the protein. Another change from denaturing is solubility in water. A non-denatured protein will bind with water and form a true liquid protein again. Another change from denaturing is digestibility. Non-denatured proteins will result in higher protein digestibility in the species consuming the protein and thus promoting improved nutritive value.

The process disclosed in Anderson et al. attempts to yield a non-heat-denatured protein. Denaturing was a drawback to existing processes attempting to yield a particulate proteinaceous product from animal feedstock. Notably, Anderson discourages from the selection of a proteinaceous product that has a low oil (fat) and water content, because a relatively high oil content in contrast with other particulate high-protein products makes the product generally more appealing to animals and seems to allow a higher moisture content than, for example, conventional fish meal without spoilage of the product. Anderson goes on to explain that prior-art fish meals containing almost no oil will usually exhibit substantial growth of molds and the like if the moisture content is above about 10%. One such fish meal process is disclosed in Canadian Patent No. 890,866 to Lum, cited by Anderson. The Lum patent suggests an initial grinding step followed by a protein digestion process, using hexane and isopropanol solvent extraction, that is carried out at temperatures ranging from about 125° F. to 145° F. and further teaches drying the de-fatted, watery protein with a spray dryer to recover a dry protein product. These process steps will yield denatured and incomplete proteins that are not a desirable protein product in many applications.

Generally, solvent extraction can be used to increase the yield of recovered lipids (fats) and protein from biomass waste streams. However, solvent extraction produces a solvent-extracted residue which contains residual solvent. Consequently, there is a need for a method for separating lipids and proteins from biomass waste streams providing a useful product having an acceptably low residual solvent content and correspondingly low toxicity effects.

There remains a need to create energy and economically efficient systems to extract solutes such as lipids and proteins from post waste biomass materials, such as animal “DAF”, coagulated and noncoagulated, flocculated and nonflocculated animal, vegetable and fruit wastestreams and other matter. The present invention addresses these and other needs.

### BRIEF SUMMARY OF THE INVENTION

In one embodiment, the invention relates to a method of extracting and separating a bio-molecule from partially or substantially dewatered biomass. Partial or substantial bulk dewatering can be accomplished by a mechanical belt press at ambient temperatures or a vacuum dryer again at ambient temperature. These can be done in combination or separately. By dewatering at ambient temperature, the cellular water is not disturbed, thus leaving the cell wall intact and fully available for digestion and nutrition. It also insures that the amino acid profile remains intact pre and post dewatering. Additionally, it has been discovered that raising or lowering the pH after the bulk dewatering will allow additional interstitial water to flow from the biomass at roughly ambient temperatures. It is theorized that the altered pH breaks the ionic and polar bonds between the flocculant and the protein and fat molecules, thereby allowing the interstitial water to be removed at ambient temperatures. The pH adjustment can be done in the presence or without a liquefied gas solvent. The biomass comprising proteins and desired bio-molecules, such as lipids and/or carotenoids, may first be introduced into an agitator vessel where it is mixed with a liquefied gas solvent. The biomass next is passed into a filter system, such as a screw press, belt press or continuous filter process system like a centrifuge. In one exemplary embodiment, a rotary pressure filter (RPF) is used and the biomass is deposited into individual filter cells of the RPF by rotating the RPF drum. While in the individual filter cells and in the wash zone of the RPF, the biomass is contacted with additional liquefied gas solvent. The desired bio-molecules (such as but not limited to lipids) are separated from the biomass by passing the solvent through the biomass containing filter cells to obtain a solvent and bio-molecule stream through the cylindrical vessel of the RPF. In this way, bio-molecules (such as lipids) and remaining water from the biomass are extracted from the biomass, and the protein is retained in the individual filter cells as a result of the separation. The individual filter cells contain a cake of accumulated protein that is substantially moisture free and can be discharged from the RPF. Solvent may remain in the accumulated protein requiring subsequent solvent removal in a further step.

In some embodiments, the biomass is pretreated and dewatered before it is introduced into either an agitator vessel or into the RPF. The pretreatment can consist of the addition of antioxidants to stabilize the lipids and promote stability of the protein to be extracted. The dewatering process may comprise partial or substantial bulk dewatering can be accomplished by a mechanical belt press at ambient temperatures or a vacuum dryer again at ambient temperature. These can be done in combination or separately. Additionally, it has been discovered that raising or lowering the pH after the bulk dewatering will allow additional interstitial water to flow from the biomass at roughly ambient temperatures and then contacting the biomass with one or more solvents and recovering the water and solvent. The solvent of the pretreatment may comprise additional quantities of the same liquefied gas solvent used within the rotary pressure filter.

In some embodiments, the method further comprises conveying the liquefied gas solvent, water and bio-molecules (such as lipids) to a distillation system where the constituents are separated. The distilled solvent is re-condensed, recovered and conveyed back into the system for re-use, either in the pretreatment unit or in the rotary pressure filter. Where the bio-molecules are lipids, the lipids may further be treated with an antioxidant to prevent spoilage, the treatment occurring, for example, after pretreatment and/or after distillation. The lipids may thereafter be stored in a vessel to be transported for sale. The water is separated using any suitable method known in the art and stored for disposal or re-use.

In some embodiments, the method further comprises mechanically filtering the dry protein, and storing the filtered protein for transportation and sale. Minimal dry waste from the protein filtration process is stored for transportation to disposal.

Another embodiment comprises a system for extracting and separating a bio-molecule from biomass. The system includes a filter system, such as a screw press, belt press, centrifuge or a rotary pressure filter with a plurality of individual filter cells distributed on a rotational drum of the rotary pressure filter. The filter system is adapted to receive a mixture or slurry of a biomass with at least one liquefied gas solvent, and with at least one mixing location through which liquefied gas solvent may be introduced so as to filter through the mixture or slurry to extract desired bio-molecule(s) and leave a filter cake comprising primarily protein. In a preferred embodiment, a heater preheats the biomass to a temperature above room temperature before introducing the biomass into the filter system. In yet another preferred embodiment, desired bio-molecules (such as lipids) extracted from the biomass and the liquefied gas solvent are recovered via at least one distillation column.

In some embodiments, the biomass is plant matter, such as but not limited to, soybean, rapeseed, canola, camolina, corn, sunflower, palm, jatropha, corn germ, distillers grains, safflower, cottonseed, flax, peanut, sesame, olive and/or coconut. The biomass also can be nuts and/or seeds, or can be other fruit and/or vegetable matter. In some embodiments, the biomass is algae.

The biomass also can be the waste stream that comes from vegetable processing for example from carrots, kale, or tomato processing. In other embodiments, the biomass is water saturated hydrocarbon waste streams or activated feedstock that comes from industrial or municipal wastewater processing.

In some embodiments, the biomass is animal matter, such as but not limited to animal by-products from a meat processing plant or by-products of wastewater from a protein processing facility. Usually, the animal matter is inedible by humans but edible to domesticated animals (e.g., canines or felines), or farm animal feed or is waste from the processing of edible animal matter. In some embodiments, the animal matter is from, for example, avian (e.g., chicken, turkey, duck, goose, ostrich, emu), porcine, bovine, ovine (e.g. lamb, sheep or goat), deer (i.e., venison), buffalo and/or fish slaughter, hatchery or meat processing. In some embodiments, the animal matter is beef rendering, chicken rendering, pork rendering, or fish rendering effluent. In some embodiments, the animal matter is poultry, pork, beef or bovine, veal, lamb and/or mutton. Bovine includes animals of the cattle group, which also includes buffalo and bison. In further embodiments, the animal matter is a flocculated or nonflocculated effluent wastewater stream from an animal processing plant, such as from poultry, beef, pork, fish and dairy processing, or the waste that arises from poultry hatchery operations, such as spent/unfertilized/broken eggs, deceased chicks or the fines that arise out of feed processing for all animal sources. In further embodiments, the animal matter may be dairy waste, such as spilled or spoiled milk and dairy process wastewater.

Suitable solvents comprise liquified gases. In some embodiments, the liquefied gas solvent is selected from butane, isobutane, propane, carbon dioxide, dimethyl ether, methane, ethane, nitrous oxide, propylene, isobutene, ethylene, sulfur hexafluoride, ammonia, gaseous hydrocarbons, gaseous halogenated hydrocarbons, fluorocarbons, sulfur dioxide, and mixtures thereof. Alternatively, co-solvents such as low molecular weight alcohols, blended dimethyl ether and/or ethanol may be used. Suitable co-solvents include, but are not limited to ethanol, propanol, isopropyl alcohol, 2-methyl-2-propanol and mixtures thereof.

In an exemplary embodiment denaturing of the protein does not occur and whole (or complete) proteins are produced because vacuum and/or pressure are utilized to perform the dewatering of a slurry in the presence of one or more solvents, without the use of high temperatures. Vacuum may also be used to decrease the boiling point of bulk or interstitial water and/or solvent which may reduce temperature requirements and avoid processing within denaturing conditions. Full vacuum is a condition wherein the internal absolute pressure is 0 kPa. Partial vacuum is a condition wherein the internal pressure is below atmospheric pressure, or about 100 kPa, 1 bar or 14.7 psi and above full vacuum. In an exemplary embodiment, the dewatering step of the process is performed under partial or full vacuum and temperatures as low as 50° and as high as 200° F. The addition of base, such as sodium or potassium hydroxide, or acid such as sulfuric or citric acid, in a concentration of about 0.1%-3% by weight can also be used for the low temperature interstitial dewatering by reducing the ionic and polar bond strength between the flocculant and the protein and lipid molecules of the biomass material and therefore reduce the energy required to bring about substantial dewatering. This low temperature and low pressure dewatering step is not only energy efficient but is useful to avoiding denaturing of the resultant proteins to be recovered by the process. Denaturing of the proteins is undesirable for high-quality protein product and is an unfortunate circumstance in prior art processes which exposure the proteins to high temperature in-process conditions.

The exemplary method of extracting and separating a bio-molecule from partially or substantially dewatered biomass as described herein produces a biomass-derived protein compound that is high in protein concentration and may have a reduced fat concentration. The exemplary method of extracting and separating a bio-molecule separates and isolates the proteins from the fats and therefore, a desired concentration of fats may be produced. In addition, the biomass-derived protein compound may have a low water concentration of no more about 20%, or a very low water concentration of no more than about 3% water, by weight. Furthermore, the exemplary mass-derived protein compound may be produced from more than one source organism, including various portions of a single type of animal, such as muscle, feathers, skin, bone, blood, or organs, or two or more different types of organisms, such as a poultry and bovine or cow. The feed stream to the method may be a commingled stream and the exemplary method produces a biomass-derived protein compound that is suitable for meal and may be hypoallergenic

An exemplary a biomass-derived protein compound may contain a substantial portion of protein molecules that are whole or complete proteins, that is, a protein molecule that contains all nine of the essential amino adds (histidine, isoleucine, leucine, lysine, methionine, phenylalanine, threonine, tryptophan, and valine) necessary for the dietary needs of humans or other animals. A substantial portion of protein molecules is at least about 65% of the protein molecules, preferably at least about 75% of the protein molecules, and more preferably at least about 85%. Conventional separating and extraction methods that utilize intensive mechanical grinding or intensive heat and pressure conditions, break down whole proteins and thus will not yield desired whole protein molecules. Exposure to high temperatures, such as greater than about 100° C. can damage or destroy the amino acids of the protein molecules, thereby producing a protein compound that is not a whole protein. Moreover, grinding and similar processes to reduce the particle size of the biomass is technique that destroys whole proteins.

An exemplary biomass-derived protein compound may contain a substantial portion of protein molecules that are non-denatured. A substantial portion of protein molecules is at least about 65% of the protein molecules and more preferably at least about 85%. Denaturation is a process in which proteins or nucleic acids lose the quaternary structure, tertiary structure and secondary structure which is present in their native state, by application of some external stress or compound such as a strong acid or base, a concentrated inorganic salt, an organic solvent (e.g., alcohol or chloroform), radiation or heat. Conventional processes for extracting and separating that use heat typically denature the proteins. Denaturing of proteins starts at about 105° F. (41° C.) and continues to about 250° F., at which point the will be denatured.

An exemplary biomass-derived protein compound may contain a substantial portion of protein molecules that are enzymatically digested, such as at least about 65%, or more preferably at least about 90%. The exemplary separating and extraction method described herein, produces a protein that is naturally digestible in the 75-80% range. In addition, enzymes may be added to increase this concentration of naturally digestible proteins to more than 80% such as 90% or more. These enzymes may be added into the continuous process as the raw material is collected or during various points in the dewatering process. Enzymatic concentration varies from 0.1% to 1% by weight have proven effective in increasing digestibility to 90% or more.

An exemplary biomass-derived protein compound has a pH that is neutral, or a pH between about 6 and 8. When a basic chemical is used in the dewatering process, as described herein, it neutralizes the acidic feedstock, thereby producing a protein compound that is neutral. The unique dewatering method enables a neutral pH of the product of the process. Likewise, some coagulant and flocculation chemistries operate under basic conditions, so the addition of an acid will neutralize the basic feedstock.

The fat concentration and protein concentration of the biomass-derived protein compound may be controlled since the fats and proteins are separated and isolated from each other. A low-fat concentration meal is often desired wherein the biomass-derived protein compound has a fat concentration of no more than about 20%, or no more than about 10% fat. A low-fat concentration biomass-derived protein compound can be produced from a biomass feedstock that has a relatively high fat concentration, such as greater than about 40% fat, or greater than 60% fat, by weight. The protein concentration may be relatively high, such as greater than 60% by weight and may be as high as 80% to more than about 90% by weight.

An exemplary biomass-derived protein compound contains a low concentration of an anti-oxidant, such as less than about 2% and preferably less than about 1%. Typically concentrations may be from about 0.1% to about 1% by weight. The antioxidant is added to prevent free fatty acid formation and rancidity in the fat as well as suppressing bio contaminant activity of a properly dewatered protein.

An exemplary biomass-derived protein compound may contain a low concentration of water, such as less than about 10% by weight and in some preferred cases less than 5% by weight. The water is removed through solvent extraction in the rotary pressure filter.

The biomass feedstock may comprise or consists of one or more organisms, such as various protein sources from a single animal, including muscle, skin, feathers, organs, and the like, or proteins from two more different animals including poultry, pork, beef, lamb, fish and the like. In addition, the biomass feedstock may comprise plant sources including grains, legumes and the like.

An exemplary biomass-derived protein compound may have an average particle size of no more than about 1,700 microns, no more than about 1000 microns, or no more than about 500 microns. The particle size may be dependent on the biomass feedstock and processing through the exemplary separation and extraction method described herein.

### DESCRIPTION OF THE DRAWING

Other aspects and advantages will be apparent from the following description given hereinafter referring to the attached drawing.

FIG. 1 is a simplified process flow diagram of biomass separation and extraction process.

FIGS. 2 to 5 show a top view diagram of an exemplary rotary pressure filter.

### DETAILED DESCRIPTION OF PREFERRED EMBODIMENTS

Rotary pressure filters are known in industry for separating suspensions, such as cellulose products, intermediate plastic products, organic chemicals, agrochemicals, instant coffee, starch, pharmaceuticals and dyes/pigments. A rotary pressure filter is a continuously operating filter having a pressure-tight design. It consists essentially of a metallic filter drum that rotates at a regulated continuous speed, an associated control head, and a metallic, pressure-rated housing. The annular space between the filter drum and the housing is sealed at the sides by means of stuffing boxes or other sealing systems. The housing is divided radially into pressure-tight chambers by means of zone separators which are held at a constant force against the drum. The surface of the drum comprises individual filter cells which are connected via outlet tubes to the control head. A detailed description of a representative rotary pressure filter may be found in WO 02/100512 A1.

When using a rotary pressure filter, a suspension to be filtered is fed continually under a constant admission pressure into the filtration zone of the rotary pressure filter and into individual filter cells. A filter cake is built up in each of the filter cells of the rotating drum. The filter cake is then conveyed into the subsequent chambers of the rotary pressure filter for after-treatment, e.g., washing and/or treatment with steam, an inert drying gas or heated solvent gas. The filter cake is taken off in an unpressurized zone of the filter either by means of an automatically operating, adjustable, mechanical scraper or/and by means of a targeted reverse pulse, typically of compressed air, nitrogen or steam. A description of the zone separators for one example of a rotary pressure filter is provided in WO 02/100512 A1.

Heretofore, continuous process filter systems have not been used to process biomass materials at pressure. Disclosed herein is a method of extracting and separating a bio-molecule, such as a lipid and/or a protein, from partially dewatered or substantially dewatered biomass that includes the step of contacting the biomass with compressed liquefied gas solvent while the biomass is moved through the continuous process filter system. After the biomass is contacted with the compressed liquefied gas solvent, a continuous stream of extracted desired bio-molecules (such as lipids) is entrained in a solvent stream that is directed out of the filter system, and a filter cake of protein is left. The invention provides a robust, scalable, low-cost process for separating water and desired bio-molecules (such as lipids) from the protein(s) of a partially dewatered or substantially dewatered biomass while maintaining desired characteristics of the protein(s) and lipids extracted.

Applicants have found that compressed gas solvents are advantageous for extracting and separating lipids from partially dewatered or substantially dewatered biomass using a rotary pressure filter. In some embodiments, the liquefied gas solvent is selected from butane, isobutane, propane, carbon dioxide, dimethyl ether, methane, ethane, nitrous oxide, propylene, isobutene, ethylene, sulfur hexafluoride, ammonia, gaseous hydrocarbons, gaseous halogenated hydrocarbons, fluorocarbons, sulfur dioxide, and mixtures thereof. In some embodiments, the liquefied solvent gas is dimethyl ether, butane or propane. Alternatively, co-solvents such as low molecular weight alcohols, blended dimethyl ether and/or ethanol may be used. Suitable co-solvents include, but are not limited to, ethanol, propanol, isopropyl alcohol, 2-methyl-2-propanol or mixtures thereof.

One preferred compressed liquefied gas solvent is liquid dimethyl ether. Dimethyl ether (also known as methyl ether) is soluble in water, and also dissolves water. This solubility is maintained along the entire vapor-pressure curve of dimethyl ether from about −5° C. to above its critical temperature (T_{c}) of 126.9° C. Dimethyl ether may be used as a solvent for the biomass material and pressures of up to 1 bar or more, 2 bars or more, and about 3 bars or more may be used in the processing. The biomass material may be dewatered before being combined or mixed with the Dimethyl ether solvent.

A biomass may be collected from any suitable source. For example, if the biomass is plant matter, agricultural waste or food processing waste may be collected. If the biomass is animal matter, agricultural waste or meat processing waste may be collected. The biomass as collected may comprise up to 20% to 85% water with the remainder being suspended or dissolved solids and any impurities that may exist in the waste stream.

To achieve maximum yields of the desired products, including for example lipids and proteins when processing a biomass, and at the same time maximize economic efficiency of the process, it is contemplated that a partially dewatered or substantially dewatered biomass be used. For a substantially dewatered biomass, the percentage of water is less than about 20% by mass or less. Preferably, the water content of the biomass entering the rotary pressure filter is less than 10% by mass, and most preferably less than 5% by mass.

To achieve maximum processability of the biomass and improve economic efficiency, in one embodiment, the biomass is continuously mixed in a suitably pressurized agitation vessel 4 with solvent, where the ratio of solvent to biomass is 5:1. A ratio of 4:1 is preferred, and a ratio of 3:1 is most preferred, while a ratio of 2:1 is also feasible. It is understood that the actual liquid (i.e., non-solid water and fat) percentages and solvent to biomass ratios employed are those that ensure that the mixture is still flowable or movable to be introduced into a rotary pressure filter for next processing steps.

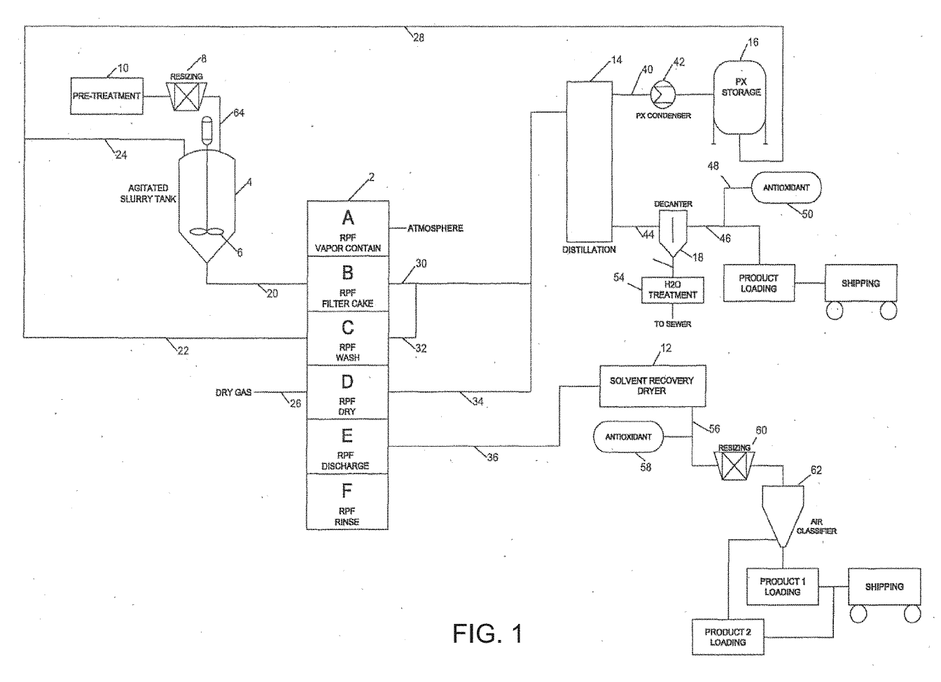

A suitable pressurized agitation vessel 4 includes, for example, a stirred tank 4 with a multi-blade impeller 6 that rotates at speeds from about 40 to about 320 revolutions per minute (see FIG. 1). The vessel interior preferably is maintained at pressures above atmospheric pressure, such as 3 to 9 bar (gage).

FIG. 1 shows one embodiment of a simplified process flow diagram of a method or process and system that can be used to separate and extract lipids and proteins from a biomass. Referring to FIG. 1, a pre-treatment unit 10 for the biomass is connected to a resizing unit 8 which is connected via a biomass supply line 64 to a pressurized agitator vessel 4 with an agitator or impeller 6. The biomass is agitated to form a slurry with a liquefied gas solvent in the agitator vessel 4.

From the agitator vessel 4 the biomass slurry is introduced into a rotary pressure filter 2. The agitator vessel 4 has at the tank inlet a solvent pipeline 24, and at the tank outlet, a pipeline 20 connected to the rotary pressure filter 2. The rotary pressure filter 2 shown in FIG. 1 is sub-divided into six working chambers A-F. The system in FIG. 1 also includes a solvent recovery dryer 12 and a distillation column 14.

From the rotary pressure filter 2, filtrate lines 30 and 32 lead to a distillation column 14. In addition, the rotary pressure filter 2 has a solvent inflow pipeline 22, an inflow line 26 for drying gas, such as Nitrogen (N2) or superheated DME vapor and an outflow line 34 for the drying gas, and a discharge chute 36 for the filter cake. Optionally, a dryer dries the filter cakes before the filter cakes are removed from each filter cell.

The discharge chute 36 is connected to the solvent recovery dryer 12. After removing any remaining solvent from the protein, the protein is discharged via a discharge chute 56 to an air classifier 62 via resizing unit 60. Leaving the outlet of the air classifier 62, the protein is transferred to packaging or other desired storage or to shipping.

The filtrate containing solvent, desired bio-molecules (e.g., lipids) and water is provided via lines 30, 32 and 34 to the distillation column 14, from which the solvent gas is removed via outlet line 40 provided with a solvent condenser 42 for the solvent. The recovered liquefied solvent is stored in solvent storage container 16 and, via pipeline 28, connected with supply line 24 to the agitator vessel 14 and with supply line 22 to the rotary pressure filter 2.

The distillation column 14 outlet is connected via outlet line 44 to a lipid/water separation unit 18, such as a decanter, from which water is removed via outlet line 52 to a water treatment unit 54. From the top of the separation unit 18, desired bio-molecules (e.g., lipids) are removed via pipeline 46 provided with antioxidant from an antioxidant storage tank 50 via pipeline 48 to packaging or other desired storage or to shipping.

The extraction and separation sequence proceeds in one preferred embodiment as follows with reference to FIG. 1: A biomass is collected from a suitable source. The biomass optionally is pretreated in pre-treatment unit 10 to remove some water. Pretreatment may include supplying thermal energy to the biomass or supplying a co-solvent to the biomass. If thermal energy is supplied to the biomass, the water content may be reduced to a level below about 20% by mass. The biomass is then supplied to the pressurized agitator tank 4 via line 64. Liquefied gas solvent also is supplied to said tank via line 24. The agitator or impeller 6, set at a rotational speed suitable for the biomass, stirs the biomass to form a mixture or slurry and ensures consistency of the biomass/solvent mixture. The mixture or slurry remains in the agitator vessel for a time sufficient to provide intimate contact between the solvent and the bio-molecules (e.g., lipids). It will be readily apparent that the mixing time varies depending on the size of agitator vessel and the flow rate of the solvent used.

The biomass/solvent mixture or slurry next is transferred from the agitator vessel 4 under constant pressure through a port 20 and into working chamber B of the rotary pressure filter 2, where the biomass mixture or slurry is deposited into the individual filter cells distributed on the rotary pressure filter's rotational drum, forming a filter cake in each filter cell. As a result of the rotary movement of the filter drum, the filter cells with the filter cakes are conveyed into working chamber C. Additional liquefied gas solvent, such as pressurized dimethyl ether or other liquefied gas solvent, is supplied to working chamber C. While a filter cake composed of protein and possibly other non-dissolvable material is forming within each of the filter cells, the filtrate, which consists principally of the remaining water and extracted bio-molecules (e.g., lipids), and solvent obtained in working chambers B and C of the rotary pressure filter by washing the biomass with the liquefied solvent, is let out of the rotary pressure filter through filtrate lines 30 and 32 and introduced into a distillation column 14. Following the washing and lipid extraction processes in chambers C and B, residual solvent content and moisture, if necessary, is adjusted in chamber D to reduce the load on subsequent processing steps. For this purpose, a drying gas at a pressure of 6 bar (gage) is supplied though inlet line 26 to drying chamber D and let out though outflow line 34 to the distillation column 14. As a result of further rotation of the filter drum, the filter cells with the dry filter cakes therein are conveyed into working chamber E where the filter cakes are forced out of the filter cells using a back pulse gas alone or in combination with a knife blade and conveyed out of the rotary pressure filter. After removal from the filter cells and the drum of the rotary pressure filter, the filter cakes, consisting of protein and any remaining solids, are taken off though the discharge chute 36 and introduced into a solvent recovery dryer 12.

In filter chamber F the filter cells are washed off to remove residual proteins and filter cake residues from the filter cells. Chamber A functions as a vapor containment zone where any gas escaping the process is captured and either discharged or recycled.

The filtrate containing solvent, extracted bio-molecules (such as lipids) and water is provided via lines 30, 32 and 34 to inlet to the distillation column 14. Overhead, the liquefied gas solvent is removed via outlet line 40 and liquefied in condenser 42. The liquefied gas solvent is stored in solvent storage container 16. From solvent storage container 16 the liquefied gas solvent may be recycled and returned for re-use via pipeline 28, connecting with supply line 24 to the agitator vessel 4 and with supply line 22 to the rotary pressure filter 2. Water and bio-molecules (such as lipids) are removed from the bottom of the column 14. Lipids and water are separated in lipid/water separation unit 18 or decanter. The lipids may be treated with an antioxidant to prevent spoilage, and stored for further processing, or may be loaded and shipped. A palatability enhancer and/or a stabilizing agent may be added to the proteins. Proteins also may be sifted for particle size classification.

The pre-treatment facility 10 optionally may be located at the same site as the rotary pressure filter 2 and other equipment for extraction and separation.

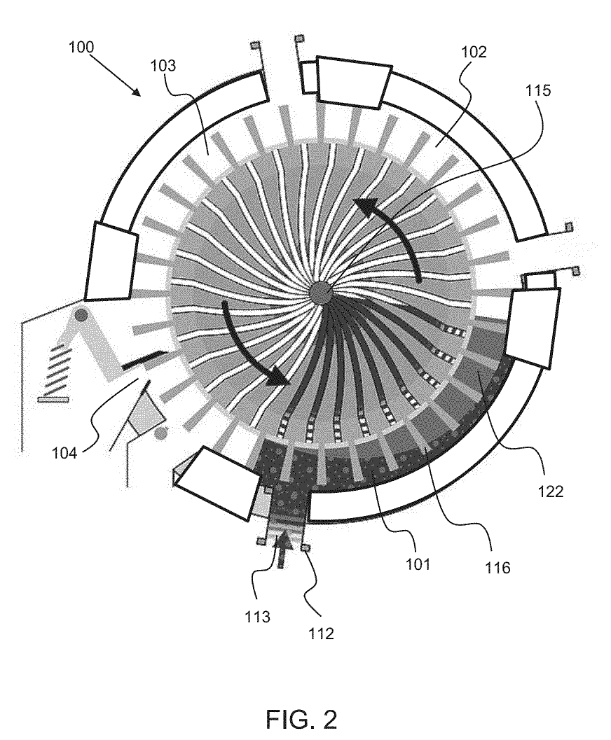

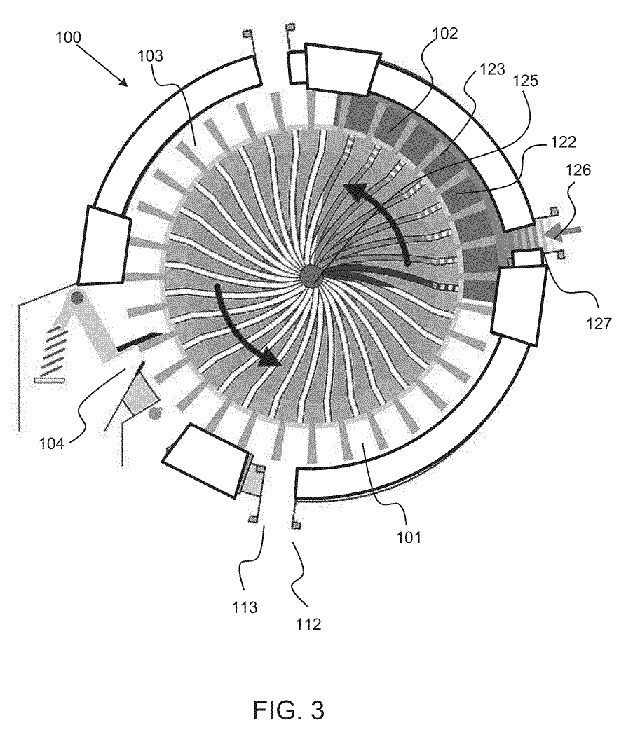

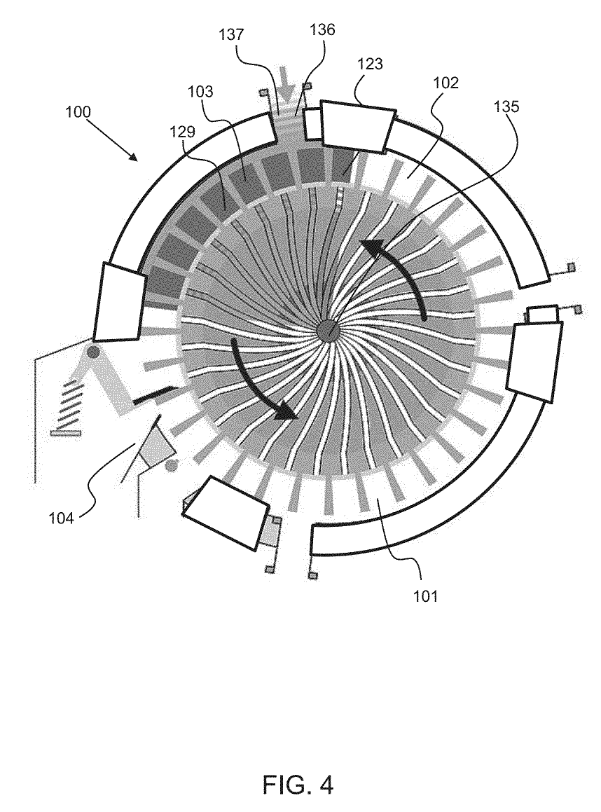

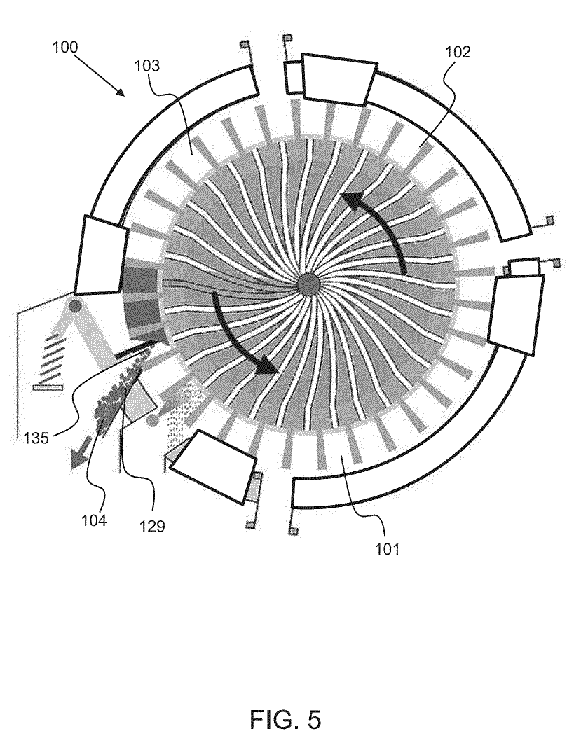

Referring now to FIGS. 2 to 5, the different regions of a rotary pressure filter system are shown with a process fluid 112 being processed therein. A rotary pressure filter has a filtration portion 101, a washing portion 102, a drying portion 103 and a discharge portion 104. As shown in FIG. 2, the first phase of the rotary pressure filter 100 is shown wherein a process fluid 112 is introduced into the rotary pressure filter through inlet 113. A liquid portion 116 of the process fluid is drawn out through outlet 115 thereby forming a filter cake 122. As shown in FIG. 3, a cake washing second phase of the rotary pressure filter 100 is shown with the cake 122 being washed with solvent 126 to form a washed filter cake 123. The solvent is introduced through inlet 127 and exits through outlet 125. As shown in FIG. 4, a washed filter cake 123 is then dried with a flow of drying gas 136, such as heated air, to form a dried filter cake 129. The drying gas enters through inlet 137 and exits through outlet 135. The dried cake 129 is then discharged through discharge outlet 147, as shown in FIG. 5.

### EXAMPLES

### Example 1

Seven (7) tons of Chicken DAF from a medium scale poultry operation are collected and pumped through the pretreatment process at a rate of 7 wet tons per hour. The solids content of the incoming DAF is on average 10% solids and 90% moisture. The moisture content after pre-treatment is on average less than 5% moisture. To thermally render the material according to the specification of the pet food industry, the material is heated to a temperature of 140° C. temperature for a minimum of 20 minutes. Alternatively, the material can be held under vacuum at 155° F. for 3 minutes or 80° F. for 6 minutes. The resultant feedstock is stored in 1 ton containers for further processing.

The feedstock is introduced into the slurry mixing tank (e.g., Silverson Rotor/Stator Mixer) at a rate of 2.2 tons per hour. The slurry tank pressure is held at 6 bar. The impeller speed is set at 8000 rpm. The solvent, liquified dimethyl ether (DME), supplied by Diversified CPC International, is added at a ratio of 3:1 by mass to the incoming feedstock. Mixing and particle size reduction is achieved through the use of a stator/rotor mixing element with an impeller speed of 8000 rpm. Further particle size reduction may be achieved through the use of an inline mixer with a speed of 8000 rpm.

The well mixed feedstock/solvent solution is then metered across a control valve to maintain a constant mass flow to the slurry inlet zone of the Rotary Pressure Filter (RPF), obtainable from BHS-Filtration Inc., Model A6. Upon entering the RPF, the feedstock/slurry mixture passes through a peek filter cloth element approximately 50 micron in opening size. The solids are deposited on the filter cloth, creating a cake thickness anywhere between 7 and 30 mm.

The cake then rotates out of the slurry inlet zone and enters the wash zone where the cake is introduced to pure liquefied DME solvent at a ratio of 2:1 based on feedstock mass. The clean solvent passes through the cake and further extracts lipids and moisture.

The slurry and wash filtrate is then stored and sent to a single distillation column for further refinement. The solvent is evaporated and collected at a purity greater than 99%. The remaining moisture and lipids pass through one distillation at a maximum temperature of 140° C. and are further separated by either standard decantation or a centrifuge. The resulting lipids have a moisture content of below 0.5%. The retained solvent in the lipids is below the 1 parts per million detectable level. Finally, the lipids are filtered in a 1 micron polyester filter cartridge, obtainable from McMaster, to remove solid particulate prior to packaging and shipment.

The protein stream leaves the RPF wash zone and goes to the RPF dry zone where nitrogen gas is passed through at a volume exchange ratio of approximately 10:1. This removes solvent down to 100% or less hold up. The drying gas is then passed through a compressor and condenser to collect the solvent for reuse.

The protein stream leaves the drying zone of the RPF and discharges to 0.5 bar discharge zone where it is removed from the RPF via scraper blade, purge gas, or both. It then travels through a pressure isolating rotary valve into a solvent dryer operating at 2 bar and a maximum temperature of 350° F. A drying gas, such as nitrogen, is passed over the protein at a ratio of 2:1 solvent to drying gas. The drying gas is then sent to a compressor and the solvent is condensed to recycle the solvent. The protein, free of solvent down to below parts per million level, is discharged from the solvent dryer through a rotary valve. From there, the protein is conveyed and may be passed through a particle size classifier so that it may be sold according to grade and product specification. In addition, stabilizing agents, such as PE-TOX from Kemin Industries (believed to be a mixture of butylated hydroxyanisole (BHA) and butylated hydroxytoluene (BHT)) or NATUROX from Kemin Industries (believed to be a mixture of tocopherol(s) with lecithin), are added to stabilize the lipid content in the protein. The lipid content in the protein stream is typically 8%, but can be as low as 3%.

### Example 2

Seven (7) tons of carrots are obtained from a carrot processing plant. The solids content of the carrots is on average 13.5% solids, and the moisture content is 86.5%. Using a Comitrol® processor available from Urschell, the carrots are resized to facilitate pumping into the slurry mixing tank. The carrot feedstock is introduced into the slurry mixing tank at a rate of 3 tons per hour. The slurry tank pressure is held at 6 bar. The solvent and co-solvent, liquified dimethyl ether (DME), supplied by Diversified CPC International, and ethanol, supplied by Sigma Aldrich, are added at a total solvent ratio of 3:1 by mass to the incoming feedstock. The ratio of DME to ethanol is 1:10. Mixing and particle size reduction is achieved through the use of a stator/rotor mixing element with an impeller speed of 8000 rpm. Further particle size reduction may be achieved through the use of an inline mixer at a speed of 8000 rpm.

The well mixed feedstock/solvent slurry is then metered across a control valve to maintain a constant mass flow to the slurry inlet zone of a Rotary Pressure Filter from BHS-Filtration Inc., Model A6. Upon entering the RPF, the feedstock/slurry mixture passes through a PEEK filter cloth element approximately 50 micron in opening size. The solids are deposited on the filter cloth creating a cake thickness anywhere between 7 and 30 mm. The cake then rotates out of the slurry inlet zone and enters the wash zone where the cake is introduced to pure liquefied DME and ethanol at a total solvent ratio of 1:1 based on feedstock mass. The clean solvent passes through the cake and further extracts lipids and moisture.

The slurry and wash filtrate is then stored and sent to a single distillation column for further refinement. The solvent is evaporated and collected at a purity greater than 99%. The remaining moisture and lipids pass through two distillation columns at a maximum temperature of 80° C. and are further separated by either standard decantation or a centrifuge. The resulting lipids have a moisture content of below 0.5%. The retained solvent in the lipids is below 10 parts per million level. Finally, the lipids are filtered in a 1 micron filter cartridge to remove solid particulate prior to packaging and shipment.

The protein stream leaves the RPF wash zone and goes to the RPF dry zone where nitrogen gas is passed through at a volume exchange ratio of approximately 10:1. This removes solvent down to 100% or less hold up. The drying gas is then passed through a compressor and condenser to collect the solvent for reuse. The protein stream leaves the drying zone of the RPF and discharges to 0.5 bar discharge zone where it is removed from the RPF via scraper blade, purge gas, or both. It then travels through a pressure isolating rotary valve into a solvent dryer operating at 2 bar and a maximum temperature of 350° F. A drying gas is passed over the protein at a ratio of 2:1 solvent to drying gas. The drying gas is then sent to a compressor and the solvent is condensed to recycle the solvent. The protein, free of solvent down to below 10 parts per million level, is discharged from the solvent dryer through a rotary valve. From there, the protein is conveyed and may be passed through a particle size classifier so that it may be sold according to grade. In addition, stabilizing agents, such as PE-TOX from Kemin Industries (believed to be a mixture of butylated hydroxyanisole (BHA) and butylated hydroxytoluene (BHT)) or NATUROX from Kemin Industries (believed to be a mixture of tocopherol(s) with lecithin), are added to stabilize the lipid content in the protein. The lipid content in the protein stream is typically 8%, but can be as low as 3%.

Numerous characteristics and advantages have been set forth in the foregoing description, together with detail of structure and function. The novel features are pointed out in the appended claims. The disclosure, however, is illustrative only, and changes may be made in detail, especially in matters of size, shape, and arrangement of parts, within the principle of the invention, to the full extent indicated by the broad general meaning of the terms in which the appended claims are expressed. Therefore, the invention must be measured by the claims and not by the description of the examples or the preferred embodiments.

What is claimed is:

1. A biomass-derived protein compound comprising:
a mixture of protein molecules from more than one source organism wherein the mixture contains at least 65% whole, non-denatured protein molecules by weight, less than 15% fat by weight, and less than 10% water by weight.

2. The biomass-derived protein compound of claim 1, wherein at least 75% of the protein molecules in the compound contain each of the following essential amino acids: histidine, isoleucine, leucine, lysine, methionine, phenylalanine, threonine, tryptophan, and valine.

3. The biomass-derived protein compound of claim 1, wherein the biomass-derived protein compound has a neutral pH with a pH between 6 and 8.

4. The biomass-derived protein compound of claim 1, wherein a substantial portion of the protein molecules are enzymatically digested.

5. The biomass-derived protein compound of claim 1, wherein the biomass from which the protein compound is derived contains greater than 50% fat by weight.

6. The biomass-derived protein compound of claim 1, further comprising an anti-oxidant in a concentration of less than 1% by weight.

7. The biomass-derived protein compound of claim 1, wherein the mixture contains at least 55% protein by weight.

8. The biomass-derived protein compound of claim 1, wherein the mixture contains less than 20% fat by weight.

9. The biomass-derived protein compound of claim 1, wherein the mixture contains less than 10% water by weight.

10. The biomass-derived protein compound of claim 1, wherein the organism source is poultry.

11. The biomass-derived protein compound of claim 1, wherein the organism source is fish.

12. The biomass-derived protein compound of claim 1, wherein the organism source is dairy.

13. The biomass-derived protein compound of claim 1, wherein the organism source is porcine.

14. The biomass-derived protein compound of claim 1, wherein the organism source is bovine derived.

15. The biomass-derived protein compound of claim 1, wherein particle size less than or equal to 1700 microns.

16. The biomass-derived protein compound of claim 6, wherein particle size less than or equal to 1000 microns.

17. The biomass-derived protein compound of claim 3, wherein the average particle size is between 250 and 750 microns.

18. A particulate proteinaceous product comprising:
a mixture of protein molecules from more than one source organism wherein the product contains at least 65% whole, non-denatured protein by weight, less than 15% fat by weight, and less than 8% water by weight.

19. The particulate proteinaceous product of claim 18, wherein the whole proteins contain nine essential amino acids in an effective proportion for dietary needs of humans or animals.

20. The particulate proteinaceous product of claim 18, wherein the non-denatured proteins maintain a native state having a quaternary structure, a tertiary structure and a secondary structure.
